(19) United States
(12) Patent Application Publication
(10) Pub. No.: US 2008/0119947 A1
(43) Pub. Date: May 22, 2008

(54) Annular Ring Implant

(75) Inventors: James Huckle, North Yorkshire (GB); Saad Ali, York (GB)

Correspondence Address: NORMAN F. HAINER, JR.;SMITH & NEPHEW, INC., 150 MINUTEMAN ROAD, ANDOVER, MA, 01801, US

(73) Assignee: Smith & Nephew, Inc., Andover, MA (US)

(21) Appl. No.: 11/941,231

(22) Filed: Nov. 16, 2007

(30) Foreign Application Priority Data
Nov. 18, 2006 (GB) GB0623065.0

(51) Int. Cl. A61F 2/02; A61B 17/58

(52) U.S. Cl. 623/23.72; 606/60

(57) ABSTRACT

The present disclosure relates to an implant having a substitute tissue structure and an annular anchoring element for anchoring the substitute tissue structure into an annular groove formed in an underlying tissue at or near an implantation site. At least a part of the annular anchoring element is bonded to the substitute tissue structure. A method of tissue repair is also disclosed.

### CROSS-REFERENCE TO RELATED APPLICATIONS

This application claims the benefit of provisional application No. GB0623065.0, filed on Nov. 18, 2006. The disclosure of this prior application is incorporated herein by reference in its entirety.

### BACKGROUND OF THE INVENTION

1. Field of the Invention

The present disclosure relates to the repair and replacement of damaged cartilage.

2. Related Art

Articular cartilage is a highly organized avascular tissue composed of chondrocytes embedded within an extracellular matrix of collagens, proteoglycans and non-collagenous proteins. Its primary function is to enable the smooth articulation of joint surfaces, and to cushion compressive, tensile, and shearing forces. Hyaline cartilage has one of the lowest coefficients of friction known for any surface to surface contact. Cartilage is frequently injured, often as a result of sports related trauma, but due to its avascular nature, articular cartilage has very limited capacity for repair. It is well known that the capacity of articular cartilage for repair is limited. Partial-thickness defects in the articular cartilage do not heal spontaneously. Injuries of the articular cartilage that do not penetrate the subchondral bone do not heal and usually progress to the degeneration of the articular surface. Injuries that penetrate the subchondral bone, and hence the vasculature, undergo repair through the formation of fibrocartilage. Although fibrocartilage fills and covers the defect, this is considered sub-optimal tissue from the biomechanical standpoint. The fibrocartilage is made to resist tension forces, while the hyaline cartilage is made to resist compression forces, to enable smooth articulation, and to withstand long-term variable cyclic load and shearing forces.

Defects in articular cartilage associated with trauma and osteochondritis dissecans represent a difficult challenge for surgeons. Focal articular cartilage defects, often found in young adults, have been increasingly recognized as a cause of pain and functional problems. The patient can expect to face progressive deterioration over time leading to advanced osteochondritis, arthritis, and the possibility of joint replacement. The knee as a weight-bearing joint is particularly susceptible to this problem, although similar injuries to the articular cartilage of other joints in humans also occur with regularity. As a result, there is a need for a minimally invasive procedure that restores the smooth and continuous articular surface with equivalent durability to the native hyaline cartilage.

The principal goals for surgical management of the symptomatic chondral and osteochondral defects are to reduce symptoms, improve joint congruence by restoring the joint surface with the best possible tissue, and to prevent additional cartilage deterioration. There are a variety of options currently available to treat and repair damaged articular cartilage, which are discussed in turn below.

Patients with relatively small cartilage defects can be treated with either anti-inflammatory medications, intra-articular steroid injections, intra-articular viscosupplements (hyaluronic acid), nutraceuticals (glucosamine or chondroitin sulfate), physical therapy, or activity modifications to alleviate their symptoms. Unfortunately, none of these treatment modalities results in cartilage healing. They may only decrease the associated pain or swelling.

One particular treatment is debridement and lavage, which is typically reserved for lower demand older patients with small lesions (less than about 2 cm² to about 3 cm²) and limited symptoms who would have difficulty with activity or weight-bearing restrictions post-operatively. It entails arthroscopic surgery where two to three small incisions are placed about the knee to place a small camera and instruments inside the joint to evaluate and treat the lesions. Loose chondral flaps that can cause mechanical symptoms are removed. Relief from this type of procedure may be incomplete or temporary because no attempt has been made to restore or repair the cartilage lesion. The recovery time from this type of procedure is relatively short, with immediate full weight-bearing and unrestricted activities.

Another technique for treating cartilage defects is by simple smoothing chondroplasty using small arthroscopic hand instruments to remove the loose fragments of articular cartilage. Subsequently, the area and edges may be smoothed after removing the loose and useless fragments of the surface using a mechanized shaver. Abrasion chondroplasty utilizes a high-speed endoscopic burr to resect a damaged area of cartilage to bleeding subchondral bone. This allows a blood clot to form over the defect which develops into a repair tissue of fibrocartilage that is relatively thin and tends to deteriorate overtime. Although this procedure has been widely used over the last two decades, the long term results are poor since the resulting fibrocartilage surface cannot support long term weight bearing, particularly in high activity patients, and is prone to wear.

Patients with small to moderate-sized lesions (about 1 cm² to about 5 cm²) and moderate demands may be treated with marrow-stimulating techniques, such as reparative subchondral bone microfracture. When a patient has a small area of damaged cartilage (i.e. not widespread knee arthritis), microfracture may be performed in an attempt to stimulate new cartilage growth. The treatment involves a disruption of subchondral bone in an attempt to induce bleeding and to initiate primitive stem cell migration from the bone marrow into the cartilage defect site. These techniques utilise primitive stem cells, which are capable of differentiating into bone and cartilage under the influence of various biologic and mechanical intra-articular factors. The subchondral bone is penetrated in order to reach a zone of vascularisation, stimulating the formation of a fibrin clot containing pluripotential stem cells. This clot differentiates and remodels, resulting in a fibrocartilaginous repair tissue. Although fibrocartilage often appears to offer the patient significant pain relief, this tissue lacks several key structural components to perform the mechanical functions, as a wear-resistant and as a weight-bearing surface. The fibrocartilage repair tissue does not produce a proper compressive stiffness against applied mechanical load and thus is subjected to an excessive deformation under physiological loading. This in turn causes a mechanical failure of the repaired tissue and eventually leads to a recurrence of degeneration of the repaired cartilage. Results for this technique are similar to abrasion chondroplasty.

Restorative osteochondral autograft transplantation (OATS) is another surgical technique that can potentially restore the height and the shape of articulating surface in focal osteochondral defects, with composite autologous material that contains all necessary ingredients: hyaline articular cartilage, intact tidemark, and a firm bone carrier. These osteochondral autograft plugs are most commonly transplanted to symptomatic lesions involving the femoral condyles. The lesions should be small to medium-sized (about 0.5 cm² to about 3 cm²) because the amount of donor tissue available is limited. The main problem with this reconstructive technique is the limited availability of autografts, which significantly reduces the choice of treatable defects down to a small focal chondral defect, and a long term donor morbidity in multiple donor sites. Deep and large, crater-like osteochondral defects are not suitable for osteochondral autograft transplantation, mainly because of the limited availability of autologous osteochondral grafts. In addition, the procedure is also technically difficult, as all grafts must be taken with the axis of the harvesting coring drill being kept perpendicular to the reticular surface at the point of harvest. Also, it is difficult to reconstruct the subchondral bone and restore the contour of the defect area, and to cover the entire defect area with hyaline articular cartilage. The dead spaces between circular grafts, the lack of integration of donor and recipient hyaline cartilage, different orientation, thickness and mechanical properties of donor and recipient hyaline cartilage are further sources of clinical concern.

Autologous Chondrocyte Implantation (ACI) is another advanced therapy which is used for intermediate to high-demand patients who have failed arthroscopic debridement or microfracture. The technique is used for larger (about 2 cm² to about 10 cm²) symptomatic lesions involving both the femoral condyles and trochlear and the patella. It allows chondrocytes to be harvested from the patients knee and cultured and multiplied. The fresh chondrocytes are then re-implanted into the patients knee and cause hyaline-like cartilage to repair the defect in articulating surface. The ACI restores the articular surface with the patients own hyaline-like cartilage without compromising the integrity of healthy tissue or the subchondral bone. This technology has demonstrated significant benefits in patients with a femoral focal lesion, in terms of diminished pain and improved function. The disadvantages of this procedure are its enormous expense. As a result, this expensive tissue engineering technology is not available in many hospitals. Furthermore, the technical complexity and need for open surgery makes it less attractive as an option for cartilage repair.

When quality of life is diminished despite the above treatments, osteotomies or total joint replacements (TJR) are historically the major surgical options, but neither of these facilitate cartilage healing. Prosthetics available for the knee joint include either total knee replacements (TKR), whereby the entire knee joint is replaced or unicompartmental knee replacements (UKR) where a single compartment of the knee joint, typically the medial condyle, is replaced. The latter treatment is a common eventuality for the patient with a large focal defect. These patients are managed with anti-inflammatory drugs, however, the remaining articular cartilage eventually erodes away resulting in pain, and loss of mobility. These problems are addressed by total joint replacement, however, the patient may face future problems associated with loosening of the implant, which may occur as a result of either wear, breakdown of the cement, osteolysis, or infection. Furthermore, healthy bone tissue has to be removed to accommodate the implant. Total joint replacement is regarded as a last resort treatment option given that the patient has to face a long and difficult recovery and rehabilitation period, and the average life span is approximately 20 years.

Cartilage replacement devices are known in the art. These devices can usually be effected immediately by surgical procedures, resulting in the alleviation of a patient's accompanying pain and also in the rehabilitation of the patient in a relatively short time span.

The use of naturally-derived (autograft, allograft or xenograft) cartilage plugs is associated with a number of problems, including lack of availability, limitations on the size of the repair that can be effected, and high potential for rejection, infection and transmission of disease.

There is therefore a need for a cartilage replacement device or graft which overcomes some or all of the problems associated with the prior art devices.

### SUMMARY OF THE INVENTION

In one aspect, the present disclosure relates to an implant. The implant includes a substitute tissue structure and an annular anchoring element for anchoring the substitute tissue structure into an annular groove formed in an underlying tissue at or near an implantation site. At least a part of the annular anchoring element is bonded to the substitute tissue structure. In an embodiment, the damaged tissue is selected from a group includes cartilage, synovium, tendon, ligament, meniscus, and bone. In another embodiment, the damaged tissue is cartilage and the annular groove is formed in subchondral bone. In yet another embodiment, the substitute tissue structure includes a material selected from the group including a natural polymer, a synthetic polymer, a ceramic material, a metal, and combinations thereof.

In a further embodiment, at least a part of the substitute tissue structure is resorbable. In yet a further embodiment, the substitute tissue structure includes at least one element selected from a group consisting essentially of an antibiotic, an analgesic, an anti-viral agent, an antimicrobial agent, an anti-inflammatory agent, a growth factor, a hormone, a cytokine, a protein, an osteogenic agent, a chondrogenic agent, a glycosaminoglycan, an immunosuppressant, a nucleic acid, a cell type, a tissue fragment, and combinations thereof. In an embodiment, the cell type is selected from a group including an osteocyte, a fibroblast, a stem cell, a pluripotent cell, a chondrocyte progenitor cell, a chondrocyte, an osteoclast, an osteoblast, an endothelial cell, a macrophage, an adipocyte, a monocyte, a plasma cell, a mesenchymal stem cell, an epithelial cell, a myoblast, a tenocyte, a ligament fibroblast, and bone marrow cell type.

In another embodiment, the tissue fragment is selected from a group including cartilage, meniscus, tendon, ligament, periosteum, and bone. In yet another embodiment, the cell type is selected from a group including an autogenic cell, an allogenic cell, a xenogenic cell, and combinations thereof. In a further embodiment, the annular anchoring element includes a material selected from a group including a natural polymer, a synthetic polymer, a gel, a ceramic material, and a metal. In yet a further embodiment, includes at least one agent selected from a group consisting essentially of an osteogenic agent, an osteoconductive agent, and an osteoinductive agent. In an embodiment, at least part of the annular anchoring element is resorbable. In another embodiment, the annular anchoring element is deformable. In yet another embodiment, the annular anchoring element is expandable.

In a further embodiment, the annular anchoring element is provided with an anti-rotational element. In yet a further embodiment, the bonding includes chemical bonding. In an embodiment, the bonding is achieved using chloroform and a polycaprolactone. In another embodiment, the substitute tissue structure includes a diameter that is larger than a diameter of the annular anchoring element.

In another aspect, the present disclosure includes a method of tissue repair. The method includes forming a groove around at least a part of the damaged tissue, wherein the groove extends into the underlying tissue below the damaged tissue; removed the tissue about which the groove extends; providing an implant including a substitute tissue structure having an annular anchoring element bonded thereto; and inserting the implant into the groove such that the annular anchoring element is located within the groove. In an embodiment, the damaged tissue is cartilage and the underlying tissue is subchondral bone.

Further areas of applicability of the present disclosure will become apparent from the detailed description provided hereinafter. It should be understood that the detailed description and specific examples, while indicating the preferred embodiment of the disclosure, are intended for purposes of illustration only and are not intended to limit the scope of the disclosure.

### BRIEF DESCRIPTION OF THE DRAWINGS

The accompanying drawings, which are incorporated in and form a part of the specification, illustrate the embodiments of the present disclosure and together with the written description serve to explain the principles, characteristics, and features of the disclosure. In the drawings:

FIG. 1 shows a perspective view of a first embodiment of the implant of the present disclosure.

FIG. 2 shows a cross-sectional view of the implant of FIG. 1 located in bone.

FIG. 3A shows a plan view of the implant of FIG. 1.

FIG. 3B shows a cross-sectional view of the implant of FIG. 3A.

FIG. 4 shows a second embodiment of the implant of the present disclosure located in bone.

FIGS. 5A-5F show implantation of the implant of FIG. 4.

### DETAILED DESCRIPTION OF THE EMBODIMENTS

The following description of the preferred embodiment(s) is merely exemplary in nature and is in no way intended to limit the disclosure, its application, or uses.

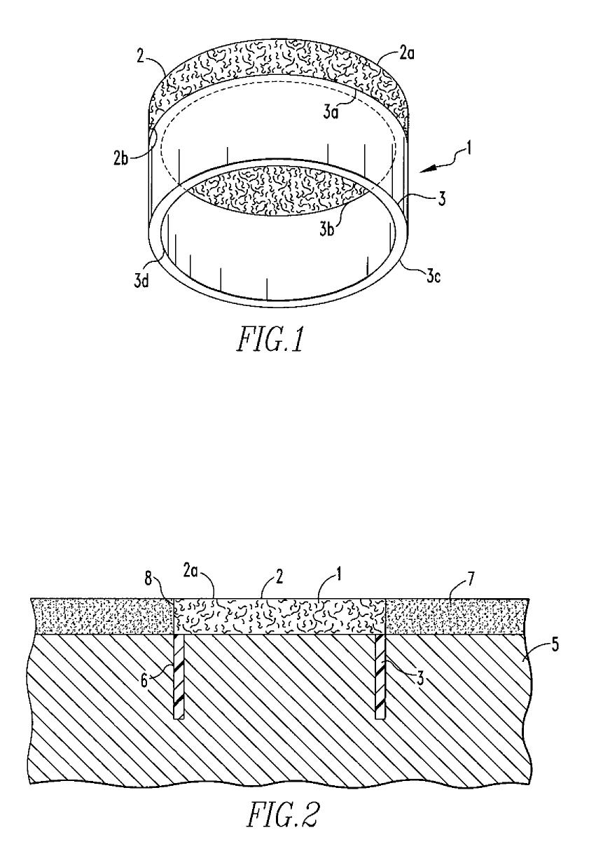

FIG. 1 shows an implant 1 according to a first embodiment of the present disclosure. The implant 1 includes a substantially circular scaffold, or substitute tissue structure 2, and an annular anchoring element 3, each having the same diameter. The scaffold 2 includes a first end 2a and a second end 2b. The annular anchoring element 3 includes a first end 3a having an opening 3b and a second end 3c having an opening 3d.

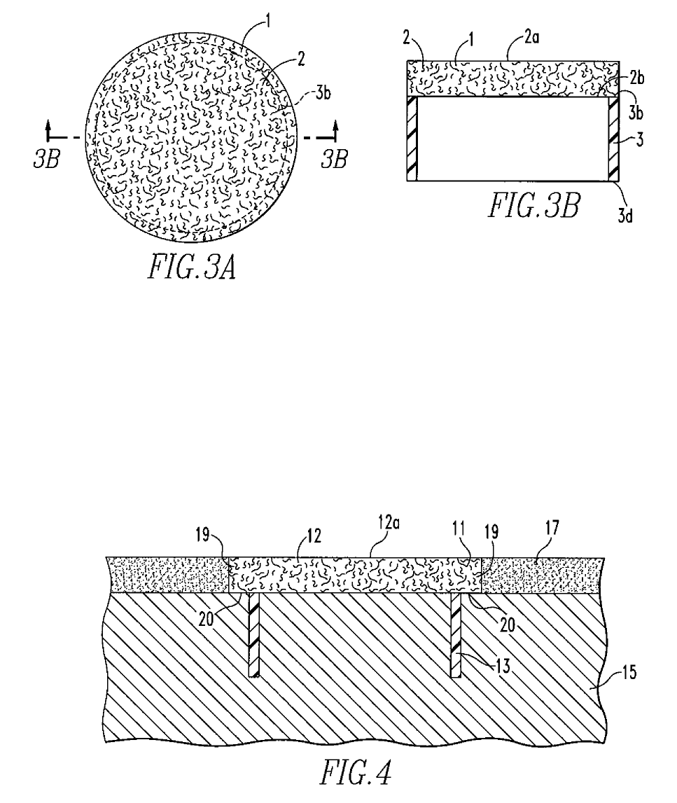

FIG. 2 shows a cross-sectional view of the implant 1 anchored into subchondral bone 5. The annular anchoring element 3 is retained within an annular groove 6 formed within the subchondral bone 5 and which surrounds a cartilage defect 8. The implant 1 is anchored into subchondral bone 5 by first forming the groove 6 around at least part of damaged cartilage and/or bone tissue (not shown). The groove 6 is formed by a reaming device or other instrument known to one of ordinary skill in the art. The damaged tissue is then removed, via a scraping device, wire brush, or other instrument known to one of ordinary skill in the art, and the implant 1 is inserted into the groove 6. The scaffold 2 is of an appropriate thickness such that when the anchoring element 3 is seated in the subchondral bone 5, the first end 2a of the scaffold 2 lies flush with the surrounding cartilage 7. FIGS. 3A and 3B show a plan view and a cross-sectional view, respectively, of the implant 1.

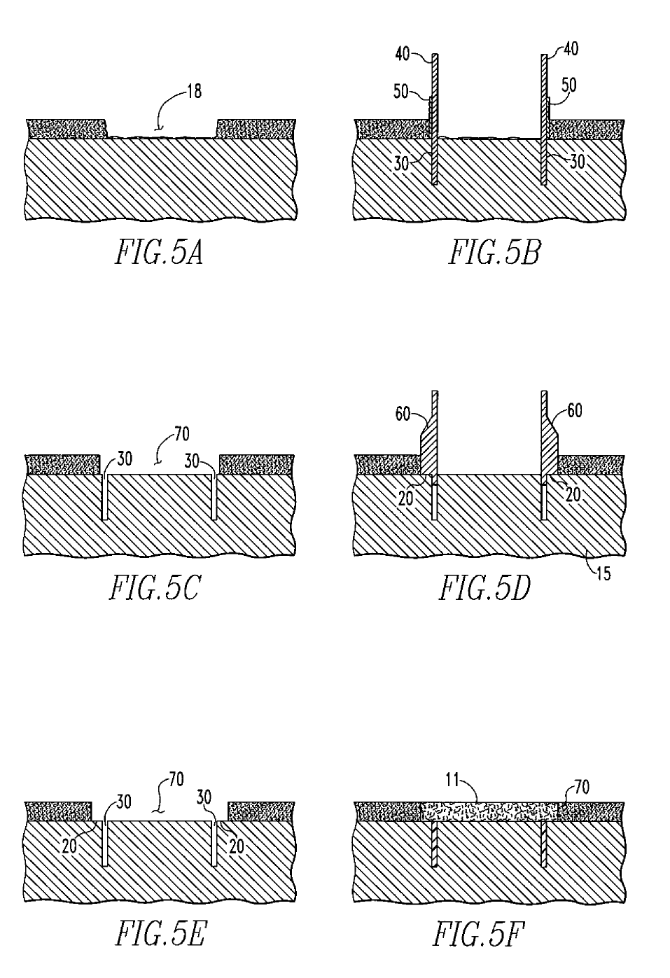

FIG. 4 shows an implant 11 according to a second embodiment of the present disclosure. This implant 11 is designed for implantation into a site of a cartilage defect, as will be further described below. The annular anchoring element 13 is retained within an annular groove 14 formed within the subchondral bone 15 and which surrounds a cartilage defect 18, as will be further described in FIG. 5. The substitute tissue structure 12 is of an appropriate thickness such that when the anchoring element is seated in the subchondral bone 15, the first end 12a of the structure 12, lies flush with the surrounding cartilage 17. The substitute tissue structure 12 has a larger diameter than the annular anchoring element 13, such that the periphery 19 of the substitute tissue structure 12 extends radially of the annular anchoring element 13. The upper surface of the subchondral bone 15 forms a ledge 20 onto which this radially extended region 19 is supported. An advantage of this region 19 is that the scaffold, or substitute tissue structure 12, is restricted from being pulled down into the groove 14.

FIGS. 5A-5F show the implantation of the implant 11. FIG. 5A shows the cartilage defect 18. FIG. 5B shows the preparation of an annular groove 30 using a saw trephine 40 and a guide 50. FIG. 5C shows the defect site 70 after preparation of the annular groove 30. FIG. 5D shows preparation of the ledge 20 of subchondral bone 15, as described above, using a cutter 60. FIG. 5E shows the defect site 70 after preparation of the ledge 20. FIG. 5F shows the implant 11 implanted into the prepared defect site 70. Other tools known to one of ordinary skill in the art may be used to prepare the annular groove 30 and the ledge 20.

The implants 1, 11 of the present disclosure are used, as described above, in the repair of tissue, such as cartilage tissue, in human or non-human animals. Formation of the grooves and removal of the damaged tissue induces bleeding of the subchondral bone and stimulates formation of a blood clot within/around the scaffold. This clot, along with the substitute tissue structure, facilitates the formation of a new tissue layer.

The macro- and microstructure of the substitute tissue structure, or scaffold, is designed to replicate structurally the tissue which it replaces. For example, when replacing articular cartilage, it is desirable that the surface of the material is contoured to mimic the surface of the natural cartilage such that there is no impingement of the implant against the apposing joint surface. The macro- and microstructure of the substitute tissue structure may also be optimized to regenerate and/or repair the desired anatomical features of the tissue that is being regenerated and/or repaired.

It is desirable that any material used within the substitute tissue structure is biocompatible. Advantageously, at least a part of the substitute tissue structure is formed of a bioresorbable material. Various parts of the substitute tissue structure may be designed to resorb at different rates. Alternatively, the rate of resorption may be isotropic across the substitute tissue structure. It is also within the scope of this disclosure that a region of the substitute tissue structure may be resorbable while another region may be non-resorbable.

The substitute tissue structure may be in the form of a solid non-deformable structure or a substantially deformable structure. In specific embodiments, the substitute tissue structure is substantially porous. Examples of suitable deformable, porous structures include, but are not limited to, felts, gauzes, gels, and sponges.

Suitable materials for the substitute tissue structure include, but are not limited to, a natural polymer, a synthetic polymer, a ceramic material, a metal, or combinations thereof. A variety of polymers can be used. As used herein the term “synthetic polymer” refers to polymers that are not found in nature, even if the polymers are naturally occurring. The term “natural polymer” refers to polymers that are naturally occurring.

In embodiments wherein the substitute tissue structure includes at least one synthetic polymer, suitable polymers include, but are not limited to, aliphatic polyesters, poly(amino acids), copoly(ether-esters), polyalkylenes oxalates, polyamides, tyrosine derived carbonates, poly(imniocarbonates), polyorthoesters, polyoxaesters, polyamidoesters, polyoxaesters containing amine groups, poly(anhydrides), polyphosphazenes, or blends thereof. Further suitable synthetic polymers for use in the invention include biosynthetic polymers based on sequences found in collagen, elastin, thrombin, fibronectin, starches, poly(amino acid), poly(propylene fumarate), gelatine, alginate, pectin, fibrin, silk oxidized cellulose, chitin, chitosan, tropoelastin, hyaluronic acid, ribonucleic acids, deoxyribonucleic acids, polypeptides, proteins, polysaccharides, polynucleotides, or combinations thereof.

In embodiments wherein the substitute tissue structure includes at least one natural polymer, suitable examples of natural polymers include, but are not limited to, fibrin-based materials, collagen-based materials, hyaluronic acid-based materials, glycoprotein-based materials, cellulose-based materials, silks, or combinations thereof. Examples of suitable bioresorbable ceramic particles include, but are not limited to, calcium sulphate, calcium phosphate, calcium carbonate, and hydroxyapatite particles.

In alternative embodiments, the substitute tissue structure comprises a non-bioresorbable material. Examples of suitable non-bioresorbable metals include, but are not limited to, stainless steel, cobalt chrome, or transition metals such as titanium and zirconium and their respective alloys. Examples of suitable non-bioresorbable ceramic particles include, but are not limited to, alumina, and zirconia. Examples of suitable non-bioresorbable polymers include, but are not limited to, polyethylene, polyvinylacetate, polymethylmethacrylate, polypropylene, poly (ethyl terephthalate), silicone, polyethylene oxide, polyethylene glycol, polyurethanes, and polyvinyl alcohol.

In further embodiments, the substitute tissue structure may further be associated with an agent that promotes healing and/or regeneration of a tissue. The term “associated” is herein defined as the agent being incorporated within, attached to, adhered to, applied to, or seeded within, at least a part of the substitute tissue structure. Suitable agents, include, but are not limited to, anti-inflammatory agents, analgesics, antibiotics, anti-viral agents, growth factors, hormones, cytokines, peptides, proteins, osteogenic agents, chondrogenic agents, anti-resprptive agents, glycosaminoglycans, immunosuppressants, nucleic acids, cells, tissue fragments, and/or combinations thereof.

The use of growth factors derived from platelet rich plasma (PRP) can enhance bone growth and maturation, graft stabilization, wound sealing, wound healing, and hemostasis. PRP concentrates a high number of autologous platelets in a small amount of plasma and mimics the last steps in the coagulation cascade, leading to the formation of a fibrin clot, which consolidates and adheres to the application site in a short period of time. Platelet alpha granules contain potent growth factors necessary to begin and substantially accelerate tissue repair and regeneration at the wound site. Growth factors shown to enhance the body's natural healing process include:
Platelet Derived Growth Factors (PDGF). PDGF initiate connective tissue healing including bone regeneration and repair. PDGF also increases mitogenesis (healing cells), angiogenesis (endothelial mitosis into functioning capillaries), and macrophage activation (debridement of the wound site and second phase source of growth factors).
Transforming Growth Factor Beta (TGF-β) increases the chemotaxis and mitogenesis of osteoblast precursors and they also stimulate osteoblast deposition of the collagen matrix of wound healing and bone regeneration.
Epidermal Growth Factors (EGF) induce epithelial development and promote angiogenesis.
Vascular Endothelial Growth Factors (VEGF) have potent angiogenic, mitogenic, and vascular permeability-enhancing activities specific for endothelial cells.

### In embodiments, PRP, platelet alpha granules, and/or growth factors derived from PRP may be applied to the substitute tissue structure.

Another class of potentially useful natural growth factors for incorporation into the substitute tissue structure are the osteogenic proteins, also referred to as bone morphogenetic, or morphogenic proteins (BMPs), which are a family of bone-matrix polypeptides which induce formation of new bone by causing the differentiation of mesenchymal cells to chondroblasts and osteoblasts.

The substitute tissue structure can also be associated with a cell. Suitable cell types include, but are not limited to, a stem cell, pluripotent cell, chondrocyte progenitor, chondrocyte, osteocyte, fibroblast, osteoclast, osteoblast, chondroblast, endothelial cell, macrophage, adipocyte, monocyte, plasma cell, mast cell, umbilical cord cell, leukocyte, stromal cell, epithelial cell, myoblast, tenocyte, ligament fibroblast or bone marrow cell type, and/or combinations thereof. In embodiments, the stem cell is a mesenchymal stem cell. In other embodiments, the substitute tissue structure is seeded with a cell population. This cell population can be of a single cell type or at least two different cell types. The cells can be seeded in a manner appropriate to the tissue that they are to form. For example, the layers of different cell types can be applied to the substitute tissue structure. Plasma treatment of the substitute tissue structure prior to or after sterilisation can be used to enhance cell adherence.

The substitute tissue structure can also be associated with at least one tissue fragment. The fragment can be derived from, for example, cartilage, meniscus, tendon, ligament, periosteum or bone, bone marrow extract, or other tissue fragments.

The cell(s) or tissue fragment(s) used may be autogenic, allogenic, xenogenic or combinations thereof.

As described above, the annular anchoring element is used to securely anchor the substitute tissue structure into a groove formed within the underlying tissue. This is of particular importance when there are moving parts adjacent to the implantation site which could dislodge the implant. In specific embodiments, the implant is for use in repairing cartilage and the element is anchored into a groove formed in the underlying bone, specifically within the subchondral bone. The annular anchoring element is of a shape that includes, but is not limited to, circular or oval. The annular anchoring element can be a continuous or non-continuous element.

It is desirable that any material used within the annular anchoring element is biocompatible. Advantageously, at least a part of the annular anchoring element is formed of a bioresorbable material. Such a material has the ability to transiently resorb, preferably in a controllable manner, within the body environment. In embodiments, various parts of the annular anchoring element may be designed to resorb at different rates. In other embodiments, the rate of resorption is isotropic across the annular anchoring element. In further embodiments, a region of the annular anchoring element may be resorbable while another region may be non-resorbable.

If the annular anchoring element is to be anchored into bone, then it is also desirable that this element includes an agent that augments bone growth. Such an agent includes, but is not limited to, an osteogenic stimulant, osteoconductive stimulant, and/or an osteoinductive stimulant. Osteogenic stimulation of bone formation refers to the stimulation of bone forming or “osteogenic” cells to form new bone growth. Osteoconductive stimulation of bone formation refers to the ability of some materials to serve as a scaffold on which bone cells can attach, migrate, grow, and divide. In this way, the bone healing response is “conducted” through the graft site. Osteoinductive stimulation of bone formation refers to the capacity of many normal chemicals in the body to stimulate primitive “stem cells” or immature bone cells to grow and mature, forming healthy bone tissue.

Suitable materials for the annular anchoring element include, but are not limited to, a natural polymer, a synthetic polymer, a ceramic material, a metal and/or combinations thereof.

In an embodiment, the annular anchoring element includes at least one synthetic polymer selected from the group including, but not limited to, aliphatic polyesters, poly(amino acids), copoly(ether-esters), polyalkylenes oxalates, polyamides, tyrosine derived carbonates, poly(imniocarbonates), polyorthoesters, polyoxaesters, polyamidoesters, polyoxaesters containing amine groups, poly(anhydrides), polyphosphazenes, and blends thereof. Suitable synthetic polymers can also include, but are not limited to, biosynthetic polymers based on sequences found in collagen, elastin, thrombin, fibronectin, starches, poly(amino acid), poly(propylene fumarate), gelatine, alginate, pectin, fibrin, silk oxidized cellulose, chitin, chitosan, tropoelastin, hyaluronic acid, ribonucleic acids, deoxyribonucleic acids, polypeptides, proteins, polysaccharides, polynucleotides, and combinations thereof.

In embodiments in which the annular anchoring element includes at least one natural polymer, suitable examples of natural polymers include, but are not limited to fibrin-based materials, collagen-based materials, hyaluronic acid-based materials, glycoprotein-based materials, cellulose-based materials, silks, and combinations thereof.

In embodiments, the annular anchoring element may be molded from polylactide carbonate (PLC), a bioresorbable polymer combined with calcium carbonate. After implantation, the PLC gradually resorbs and the calcium carbonate promotes the growth of cancellous, or porous, bone within the bone. In other embodiments, the annular anchoring element includes the bioresorbable polymer, poly (D,L-lactide-co-glycolide). After implantation the polymer gradually resorbs. In yet other embodiments, the annular anchoring element includes a bioresorbable ceramic, for example, calcium phosphate, calcium carbonate, hydroxyapatite particles, or combinations thereof.

In alternative embodiments, the annular anchoring element comprises a non-bioresorbable material. Examples of suitable non-bioresorbable metals include, but are not limited to, stainless steel, cobalt chrome, or transition metals such as titanium and zirconium and their respective alloys. Examples of suitable non-bioresorbable ceramic particles include alumina, zirconia and calcium sulphate particles. Examples of suitable non-bioresorbable polymers include polyethylene, polyvinylacetate, polymethylmethacrylate, polypropylene, poly (ethyl terephthalate), silicone, polyethylene oxide, polyethylene glycol, polyurethanes and polyvinyl alcohol.

It is further envisaged that the annular anchoring element can be made of a non-porous material, a porous material, or combinations thereof.

The annular anchoring element may be rigid and therefore pre-formed to the shape of the groove. Alternatively the element may be deformable, advantageously resiliently deformable, thereby allowing any necessary deformation of the shape of the element to correspond with the shape of the groove. Alternatively the annular anchoring element may be expandable after implantation to fit securely into the groove, for example the annular ring may be formed of a shape memory polymer.

The osteoconductive properties of the annular anchoring element may further be enhanced by texturing of at least a part of the surface, for example, by etching or grit-blasting.

In further embodiments, the annular anchoring element can be provided with an anti-rotational element, which may, for example, be a threaded ring or a barbed ring. Additional elements may be applied to or incorporated within the annular anchoring element to increase its stability within the groove, for example, a tack, a screw, a barb, a pin, or a plug.

In further embodiments, the substitute tissue structure may further be associated with an agent that promotes healing and/or regeneration of a tissue. The term “associated” is herein defined as the agent being incorporated within, attached to, adhered to, applied to, or seeded within, at least a part of the substitute tissue structure. Suitable agents, include, but are not limited to, anti-inflammatory agents, analgesics, antibiotics, anti-viral agents, growth factors, hormones, cytokines, peptides, proteins, osteogenic agents, chondrogenic agents, anti-resorptive agents, glycosaminoglycans, immunosuppressants, nucleic acids, cells, tissue fragments, and/or combinations thereof.

Although reference herein is made to the repair of damaged articular cartilage, it should be understood that the damaged tissue may be other types of tissue, for example, bone or skin, including damaged surfaces of or defects in the bone itself.

### Example

Felt & ring anchored implants (PLGA/PLC) of a resorbable non-woven felt of PLGA (10:90) attached directly to a ring of poly lactide carbonate (PLC) were made. Non-woven (un-bonded) poly(L-lactic-co-glycolic) acid (PLGA 10:90) scaffolds (TO022-150-1-11) were produced having a diameter of 7 mm, a thickness of 2 mm, a felt density of 122 mg/cc, and a porosity of 91%. Poly Lactide Carbonate (PLC 65:35) rings with a diameter of 7 mm were produced. Powdered polycaprolactone, PCL (CAPA 686, Solway) was dissolved in Chloroform (GPC Grade) to form a 6% w/v solution. The PCL solution was then introduced onto at least part of one end of the PLC ring using a small spatula. The PLGA non-woven felt is then applied to the side of the PLC ring provided with the PCL solution to bond them together. The felt & ring implants are then placed on a release paper and air-dried in a fume cupboard overnight and subsequently dried in a vacuum oven at 40° C. for 24 hours.

Felt & ring (7 mm, diameter) anchored implants (PLGA/PLC) of a resorbable non-woven felt of PLGA (10:90) attached directly to a ring of poly lactide carbonate (PLC) were produced as above, and the strength of bonding was tested by attempting to separate them physically. The bonded felt & ring implants were very robust and it was not possible to separate them.

As various modifications could be made to the exemplary embodiments, as described above with reference to the corresponding illustrations, without departing from the scope of the disclosure, it is intended that all matter contained in the foregoing description and shown in the accompanying drawings shall be interpreted as illustrative rather than limiting. Thus, the breadth and scope of the present disclosure should not be limited by any of the above-described exemplary embodiments, but should be defined only in accordance with the following claims appended hereto and their equivalents.

What is claimed is:

1. An implant comprising:
a substitute tissue structure; and
an annular anchoring element for anchoring the substitute tissue structure into an annular groove formed in an underlying tissue at or near an implantation site,
wherein at least part of the annular anchoring element is bonded to the substitute tissue structure.

2. The implant of claim 1 wherein the damaged tissue is selected from a group consisting essentially of cartilage, synovium, tendon, ligament, meniscus, and bone.

3. The implant of claim 1 wherein the damaged tissue is cartilage and the annular groove is formed in subchondral bone.

4. The implant of claim 1 wherein the substitute tissue structure includes a material selected from the group consisting essentially of a natural polymer, a synthetic polymer, a ceramic material, a metal, and combinations thereof.

5. The implant of claim 1 wherein at least a part of the substitute tissue structure is resorbable.

6. The implant of claim 1 wherein the substitute tissue structure comprises at least one element selected from a group consisting essentially of an antibiotic, an analgesic, an anti-viral agent, an antimicrobial agent, an anti-inflammatory agent, a growth factor, a hormone, a cytokine, a protein, an osteogenic agent, a chondrogenic agent, a glycosaminoglycan, an immunosuppressant, a nucleic acid, a cell type, a tissue fragment, and combinations thereof.

7. The implant of claim 6 wherein the cell type is selected from a group consisting essentially of an osteocyte, a fibroblast, a stem cell, a pluripotent cell, a chondrocyte progenitor cell, a chondrocyte, an osteoclast, an osteoblast, an endothelial cell, a macrophage, an adipocyte, a monocyte, a plasma cell, a mesenchymal stem cell, an epithelial cell, a myoblast, a tenocyte, a ligament fibroblast, and bone marrow cell type.

8. The implant of claim 7 wherein the tissue fragment is selected from a group consisting essentially of cartilage, meniscus, tendon, ligament, periosteum, and bone.

9. The implant of claim 7 wherein the cell type is selected from a group consisting essentially of an autogenic cell, an allogenic cell, a xenogeneic cell, and combinations thereof.

10. The implant of claim 1 wherein the annular anchoring element includes a material selected from a group consisting essentially of a natural polymer, a synthetic polymer, a gel, a ceramic material, and a metal.

11. The implant of claim 1 wherein the annular anchoring element comprises at least one agent selected from a group consisting essentially of an osteogenic agent, an osteoconductive agent, and an osteoinductive agent.

12. The implant of claim 1 wherein at least part of the annular anchoring element is resorbable.

13. The implant of claim 1 wherein the annular anchoring element is deformable.

14. The implant of claim 1 wherein the annular anchoring element is expandable.

15. The implant of claim 1 wherein the annular anchoring element is provided with an anti-rotational element.

16. The implant of claim 1 wherein the bonding includes chemical bonding.

17. The implant of claim 1 wherein the bonding is achieved using chloroform and a polycaprolactone.

18. The implant of claim 1 wherein the substitute tissue structure includes a diameter that is larger than a diameter of the annular anchoring element.

19. A method of tissue repair comprising:
forming a groove around at least a part of damaged tissue, the groove extending into the underlying tissue below the damaged tissue;
removing the damaged tissue about which the groove extends;
providing an implant comprising a substitute tissue structure having an annular anchoring element bonded thereto; and
inserting the implant into the groove such that the annular anchoring element is located within the groove.

20. The method of claim 19 wherein the damaged tissue is cartilage and the underlying tissue is subchondral bone.
